(19) United States
(12) Patent Application Publication
(10) Pub. No.: US 2024/0342187 A1
(43) Pub. Date: Oct. 17, 2024

(54) USE OF NEP INHIBITORS FOR THE TREATMENT OF GASTROINTESTINAL SPHINCTER DISORDERS

(71) Applicant: Curtails LLC, Smyrna, GA (US)

(72) Inventor: Mark G. Currie, Boston, MA (US)

(21) Appl. No.: 18/569,792

(22) PCT Filed: Jun. 13, 2022
(86) PCT No.: PCT/US22/33238

§ 371 Date: Dec. 13, 2023

Related U.S. Application Data
(60) Provisional application No. 63/210,185, filed on Jun. 14, 2021.

(51) Int. Cl. A61K 31/55; A61K 31/216; A61K 31/795; A61K 38/05; A61K 45/06; A61P 1/00

(52) U.S. Cl. CPC A61K 31/55; A61K 31/216; A61K 31/795; A61K 38/05; A61K 45/06; A61P 1/00

(57) ABSTRACT

The present disclosure relates to methods, uses, pharmaceutical compositions and kits comprising an NEP inhibitor or a pharmaceutically acceptable salt thereof, alone or in combination with one or more additional therapeutic agents, for the treatment of a gastrointestinal sphincter disorder. Gastrointestinal sphincter disorders include, but are not limited to, achalasia and other hypertensive or spastic sphincter disorders elsewhere in the gastrointestinal tract or sphincter spasms, or hypertensive sphincter disorders of the gastrointestinal tract.

### CROSS REFERENCE TO RELATED APPLICATION

This application claims priority under 35 U.S.C. § 119(e) to the U.S. Provisional Patent Application 63/210,185, filed Jun. 14, 2021. The entire contents of the aforementioned application are hereby incorporated by reference in their entirety.

### FIELD OF THE INVENTION

The present disclosure relates to methods of using neural endopeptidase (NEP) inhibitors and pharmaceutically acceptable salts thereof, alone or in combination with one or more additional therapeutic agents, for the treatment of gastrointestinal sphincter dysfunction or disorders, such as achalasia of the lower esophageal sphincter sphincter of the gastrointestinal tract, hypertensive sphincter disorders of the gastrointestinal tract and spastic sphincter disorders of the gastrointestinal tract.

### BACKGROUND

The human body contains more than 60 sphincters in different body systems. The gastrointestinal tract contains several important sphincters: upper and lower esophageal sphincters (UES and LES, respectively), the pyloric sphincter or pylorus (at the lower end of the stomach), the ileocecal sphincter or valve at the junction of the latest part of the small intestine (ileum) and the large intestine, which functions to limit the reflux of colonic contents back into the ileum, the sphincter of Oddi (also named Glisson's sphincter), controlling secretions from the liver, pancreas and gall bladder into the duodenum and, at the anus, two sphincters are present, which control the exit of feces from the body (internal anal sphincter and external anal sphincter). The control of the inner anal sphincter is involuntary and the control of the outer sphincter is voluntary.

Achalasia refers to the failure of circular smooth muscle fibers in the distal esophagus to relax, which causes the LES to remain closed and fail to open when needed—as in swallowing—and frequently results in the widening of the structure above the muscular constriction. Achalasia is characterized by exceedingly high values of manometric pressure of the LES. Failure of sphincter relaxation elsewhere in the gastrointestinal tract would be associated with similar abnormalities.

A sphincter is considered hypertensive when its resting pressure is elevated above normal physiological resting pressure. In achalasia, the pressure of the LES is persistently elevated and the LES fails to relax normally with swallowing. Sphincter pressure is usually measured by manometry.

For example, in achalasia of the esophagus, or simply, achalasia, the lower esophageal sphincter (LES) fails to relax upon swallowing (<75% relaxation observed) and a value higher than 100 mm Hg is obtained by manometry (less than 26 mm Hg is considered normal). Values between 26 mm Hg and 100 mm Hg fall under hypertensive LES (HTLES). HTLES is usually defined by a resting pressure measured at the respiratory inversion point on stationary manometry of greater than 26 mm Hg (ninety-fifth percentile of normal). The most common symptoms in patients with HTLES are regurgitation (75%), heartburn (71%), dysphagia (71%), and chest pain (49%). The most common primary presenting symptoms are heartburn and dysphagia.

Various treatments are available, although they are all palliative and none cures the condition. Sublingual nifedipine (a calcium channel blocker) can improve outcomes in up to 75% of people—predominantly with mild disease. These drugs are only used for patients who are considered too old or frail for more invasive treatments. Botulinum toxin (Botox) is used in some patients to produce temporary chemical denervation. However, this treatment is also generally limited to patients who are considered unfit or unsuitable for a more definitive procedure. More permanent relief is achieved by esophageal pneumatic dilatation (balloon dilatation), or surgical cleaving of the muscles of the distal esophagus (laparoscopic Heller's myotomy—LHM), or—increasingly—by an endoscopic procedure known as peroral endoscopic myotomy (POEM).

All current treatment modalities suffer from either low effectiveness (i.e., calcium channel blockers, Botulinum toxin injection) or from being initially effective but having efficacy that diminishes over time or high levels of relapse. In most cases, subsequent treatments involve cumulative risks. Therefore, there remains an unmet medical need for novel, well tolerated, and effective therapies to treat disorders involving dysfunction of gastrointestinal sphincters.

### SUMMARY OF THE INVENTION

In one aspect, the invention provides a method of treating a gastrointestinal sphincter disorder in a patient in need thereof, comprising administering to said patient a therapeutically effective amount of neural endopeptidase (NEP) inhibitor or a pharmaceutically acceptable salt thereof.

In another aspect, the invention provides a pharmaceutical composition comprising an NEP inhibitor, or a pharmaceutically acceptable salt thereof, for use in the treatment of a gastrointestinal sphincter disorder in a patient in need thereof.

In another aspect, the invention also provides a pharmaceutical composition comprising an NEP inhibitor, or a pharmaceutically acceptable salt thereof, and one or more additional therapeutic agents, for use in the treatment of a gastrointestinal sphincter disorder in a patient in need thereof.

### DETAILED DESCRIPTION

Reference will now be made in detail to certain embodiments of the invention, examples of which are illustrated in the accompanying structures and formulae. While the invention will be described in conjunction with the enumerated embodiments, it will be understood that they are not intended to limit the invention to those embodiments. Rather, the invention is intended to cover all alternatives, modifications and equivalents that may be included within the scope of the present invention as defined by the claims. The present invention is not limited to the methods and materials described herein but includes any methods and materials similar or equivalent to those described herein that could be used in the practice of the present invention. In the event that one or more of the incorporated literature references, patents or similar materials differ from or contradict this application, including but not limited to defined terms, term usage, described techniques or the like, this application controls. The compounds described herein may be defined by their chemical structures and/or chemical names. Where a compound is referred to by both a chemical structure and a chemical name, and the chemical structure and chemical name conflict, the chemical structure is determinative of the compound's identity.

### Gastrointestinal Sphincter Disorders

In one aspect, the invention provides a method of treating a gastrointestinal sphincter disorder in a patient in need thereof, comprising administering to said patient a therapeutically effective amount of neural endopeptidase (NEP) inhibitor or a pharmaceutically acceptable salt thereof.

### Gastrointestinal Sphincter Dysfunction

The gastrointestinal tract is commonly divided into several parts: mouth, throat, esophagus, stomach, small intestine and large intestine. These parts are separated from each other by special muscles called sphincters which are closed for most of the time but which relax in response to specific physiological conditions such as food ingestion or digestion. Sphincters regulate the movement of food from one part to another, and mostly unidirectionally from mouth to anus.

The human body contains more than 60 sphincters in different body systems. The gastrointestinal tract contains several important sphincters: upper and lower esophageal sphincters (UES and LES, respectively), the pyloric sphincter or pylorus (at the lower end of the stomach), the ileocecal sphincter or valve at the junction of the latest part of the small intestine (ileum) and the large intestine, which functions to limit the reflux of colonic contents back into the ileum, the sphincter of Oddi (also named Glisson's sphincter), controlling secretions from the liver, pancreas and gall bladder into the duodenum and, at the anus, two sphincters are present, which control the exit of feces from the body (internal anal sphincter and external anal sphincter). The control of the inner anal sphincter is involuntary and the control of the outer sphincter is voluntary.

An achalasia refers to the failure of circular smooth muscle fibers to relax, In achalasia, the LES remains closed and fails to open when needed (i.e., swallowing, eructation, vomiting) and eventually results in the widening (or dilation) of the esophagus above the sphincter. Abnormal contractions of sphincters of the gastrointestinal tract are characterized by abnormally high pressures (as measured by manometry) at the sphincter.

The LES may be considered hypertensive when its resting pressure after a swallow (as measured by manometry) is higher than normal but not as high as in achalasia. Since the LES still partially opens, symptoms are typically less severe than in advanced achalasia.

For example, in achalasia, the lower esophageal sphincter (LES) fails to relax upon swallowing (<75% relaxation observed) and a value higher than 100 mm Hg may be obtained at manometry (less than 26 mm Hg is considered normal). Values between 26 mm Hg and 100 mm Hg fall under hypertensive LES (HTLES). HTLES is usually defined by a resting pressure measured at the respiratory inversion point on stationary manometry of greater than 26 mm Hg (ninety-fifth percentile of normal). The most common symptoms in patients with HTLES are regurgitation (75%), heartburn (71%), dysphagia (71%), and chest pain (49%). The most common primary presenting symptoms are heartburn and dysphagia.

A spastic sphincter is one that is unable to relax normally, but may do so inappropriately—at the wrong times or for the wrong duration of time. Spasticity of a sphincter may cause pain and other symptoms related to the dysregulation of the normal movement of contents through the gastrointestinal tract.

Without a modifier or qualifier, the term “achalasia” usually refers to achalasia of the esophagus, due to a dysfunction of the LES. It is also called “esophageal achalasia”, “achalasia cardiae”, “cardiospasm” or, sometimes, “esophageal aperistalsis” (as dysfunction of the LES is associated with absence of normal esophageal body peristalsis). However, sphincter dysfunctions can happen at various points along the gastrointestinal tract; failure of relaxation of the internal anal sphincter, for instance, is Hirschsprung's disease.

Throughout this disclosure, the terms achalasia and esophageal achalasia are used interchangeably. When referring to dysfunction of gastrointestinal tract sphincters other than the LES, alternative terms will be used. For instance, other types of gastrointestinal tract sphincter dysfunction contemplated in this disclosure include those of the pyloric sphincter (non-obstructive pyloric stenosis), ileocecal valve/sphincter, spasm of the sphincter of Oddi or Glisson's sphincter (sphincter of Oddi dysfunction, SOD) and spasm of the internal anal sphincter (Hirschsprung's disease).

Esophageal achalasia is one cause of dysphagia (difficulty swallowing). It is a rare disease characterized by failure of the LES to relax in response to deglutition, and aperistalsis of the esophageal body. It is a motility disorder involving the smooth muscle layer of the esophagus and the LES. It has an annual incidence of approximately 2 in 100,000 and a prevalence rate of 10 in 100,000. It has no gender predominance.

Characteristic clinical manifestations of achalasia are difficulty swallowing solids or liquids, regurgitation of undigested food that had been retained in the esophagus, and sometimes chest pain (cardiospasm) or heartburn. In many instances these symptoms result in weight loss. Some people may also experience coughing when lying in a horizontal position. Food and liquids may be retained in the esophagus in achalasia and may be inhaled into the lungs (aspiration)—typically during sleep—possibly resulting in pneumonia. In addition, 40% of patients with achalasia report occurrence of at least one respiratory symptom, including cough, hoarseness, wheezing, shortness of breath and sore throat.

Clinical symptoms can initially manifest at any age, but usually manifest between the ages of 25 and 60. Diagnosis is based on a careful analysis of a patient's symptoms and confirmed with a combination of endoscopy, esophageal manometry (esophageal motility measurement) and barium swallow radiographic studies.

Various treatments are available, although none is curative. Sublingual nifedipine (a calcium channel blocker) improves symptoms in up to 75% of people—although, most typically, those with only mild or moderate disease. Botulinum toxin (Botox) injection into the area of the LES for chemical denervation may also be used in some patients. However, these treatments are usually reserved for patients considered to be too old or too infirm for a more definitive treatment approach. More permanent symptoms relief is achieved through esophageal pneumatic dilatation (balloon dilatation), or surgical cleaving of the muscles of the distal esophagus (laparoscopic Heller myotomy—LHM), pr—increasingly—by an endoscopic approach known as peroral endoscopic myotomy (POEM). However, each of these is highly operator-dependent and some are only available at secondary or tertiary referral centers. Each is associated with possible complications including esophageal perforation with balloon dilatation and the development of gastroesophageal reflux disease (GERD) following LHM. GERD symptoms are so common after LHM that most surgeons combine the operation with surgical enhancement of the LES (fundoplication) to reduce the tendency of development of GERD. All current treatment modalities suffer from either low effectiveness or being initially effective but having efficacy that diminishes over time or high levels of relapse. In most cases, subsequent treatments involve cumulative risks.

Manometry is the gold standard for establishing the diagnosis of achalasia. Some characteristic manometric findings of achalasia are the following: LES fails to relax upon swallows (<75% relaxation observed); resting LES pressure is abnormally elevated (<26 mm Hg is normal, whereas a value>100 is consistent with achalasia); aperistalsis of the esophageal body; relative increase in intra-esophageal pressure as compared with intragastric pressure. All patients with suspected achalasia should also undergo upper gastrointestinal endoscopy to rule out other causes, such as mechanical obstruction due to a tumor in the distal esophagus or proximal stomach (so called pseudoachalasia or malignant pseudoachalasia). Achalasia has no known cause (it is idiopathic). It is believed to be due to the loss of distal esophageal inhibitory neurons. However, a condition similar to achalasia occurs secondary to Chagas disease (an infectious disease common in Central and South America).

Values between 26 mm Hg and 100 mm Hg may be considered hypertensive LES (HTLES).

Several types of hereditary achalasia are also known. These extremely rare forms have infantile onset, usually displaying initial symptoms within 2-22 months of birth. They are associated with mutations in a single gene, for instance involving loss of function of neuronal nitric oxide synthase (nNOS) or soluble guanylate cyclase (sGC).

Although achalasia is a relatively rare condition, it carries a risk of complications, including aspiration pneumonia and esophageal cancer.

Other sphincter dysfunctions similarly carry risk of complications.

### Sphincter Dysfunction and the NO/cGMP Pathway

Postganglionic myenteric neurons of the myenteric plexus are responsible for controlling esophageal contractility. There are two populations of neurons involved in this process: excitatory neurons (using acetylcholine or Ach as the neurotransmitter) and inhibitory neurons (using nitric oxide (NO) or vasoactive intestinal peptide (VIP) as the neurotransmitter). Both types of neurons innervate the muscle of the muscularis propia and the LES. The myenteric plexus is a layer of nervous tissue situated between the two layers of smooth muscle that form the muscularis propia. Both circular and striated smooth muscle tissue form the muscularis propia of the esophageal body. The LES is formed by circular smooth muscle. LES pressure at any moment reflects the balance between excitatory and inhibitory neurotransmission. At the LES, inhibitory neurons mainly use NO as the neurotransmitter.

Achalasia is believed to be due to the loss of inhibitory myenteric neurons. In the early disease stages, myenteric neurons have been found (through tissues obtained from autopsy or surgical resection) to be surrounded by inflammatory cells. The presence of antibodies has also been considered to suggest an autoimmune mechanism. In the end stages of the disease, there is a marked depletion of myenteric ganglia and development of fibrosis. In severe cases, the myenteric nerves have been found to be almost completely replaced by collagen. Whereas at the LES, loss of inhibitory myenteric neurons is responsible for failure to relax, in the peristaltic esophageal body, achalasia is characterized by a loss of intrinsic acetylcholine-containing nerves, which leads to excessive relaxation and lack of peristalsis. However, resolving the LES issue alone often results in major symptomatic relief for the patient.

Similarly, most of the muscle along the walls and sphincters of the digestive system is smooth muscle, except for the first section of the esophagus, the UES and the external anal sphincter. Motility of the gastrointestinal tract at the smooth muscle level is controlled by the enteric nervous system through the myenteric plexus. Thus, relaxation of the sphincters situated along the gastrointestinal tracts is controlled by the tissue concentrations of nitric oxide synthesized by the neurons of the inhibitory cells of the myenteric plexus.

In cells, NO is synthesized from arginine and oxygen by various nitric oxide synthase (NOS) enzymes and by sequential reduction of inorganic nitrate. Three distinct isoforms of NOS have been identified: inducible NOS (iNOS or NOS II) found in activated macrophages; constitutive neuronal NOS (nNOS or NOS I), involved in neurotransmission, long term potentiation and gastrointestinal motility among other things; and constitutive endothelial NOS (eNOS or NOS III) which regulates smooth muscle relaxation in the vasculature, and blood pressure.

Soluble guanylate cyclase (sGC) is the primary receptor or target for NO in vivo. sGC is expressed in smooth muscle as well as other cells of the gastrointestinal tract. sGC can be activated via both NO-dependent and NO-independent mechanisms. In response to this activation, sGC converts guanosine triphosphate (GTP) into the secondary messenger cyclic guanosine monophosphate (cGMP). The increased level of cGMP, in turn, modulates the activity of downstream effectors including protein kinases, phosphodiesterases (PDEs) and ion channels.

Experimental and clinical evidence suggest that reduced availability of endogenously produced NO by inhibitory myenteric neurons contributes to the development of achalasia. For example, mice lacking neuronal NO synthase (nNOS) show achalasia-like features including LES hypertension with impaired relaxation. Consistent with this animal model, some achalasia patients have polymorphisms of genes encoding NO synthase (NOS). Low nNOS activity has also been observed in biopsies of the muscularis externa of the esophagus from achalasia patients. In addition, in a recent genetic study, nine individuals shown to have mutations leading to a loss of function of the sGC enzyme developed severe moyamoya and early-onset achalasia. The reported benefit of treatment (off-label) with nitrate donors and phosphodiesterase 5 (PDE5) inhibitors provides further evidence supporting the potential of the NO-sGC-cGMP pathway in achalasia. Both nitrates, which increase NO concentration, and the PDE5 inhibitor sildenafil, which blocks the degradation of cGMP, reduce LES pressure in achalasia patients.

NO-independent, heme-dependent, sGC stimulators, such as the ones presented in this disclosure, have several important differentiating characteristics when compared to other types of sGC modulators. These include crucial dependency on the presence of the reduced prosthetic heme moiety for their activity, strong synergistic enzyme activation when combined with NO and stimulation of the synthesis of cGMP by direct stimulation of sGC, independent of NO. The benzylindazole compound YC-1 was the first sGC stimulator to be identified. Additional sGC stimulators with improved potency and specificity for sGC have since been developed.

Thus, in patients suffering from achalasia, the augmentation of cGMP production by sGC stimulators in response to impaired NO signaling can ameliorate excessive pressure in the LES and potentially elsewhere in the esophageal body, and consequently may improve the symptoms of achalasia.

Similarly, experimental and clinical evidence supports the notion that a dysfunctional NO-sGC-cGMP pathway is the cause of many dysfunctions affecting sphincters along the GI tract, including hypertensive and spastic sphincter conditions.

Depending on the disease, the dysfunctional NO-sGC-cGMP pathway affecting different sections of the gastrointestinal tract may be the result of damage to the myenteric inhibitory neurons (thus reducing NOS expression and NO synthesis) or damage to the smooth muscle (thus reducing expression of the target of NO, the sGC enzyme) or both. In some cases, both tissues may be relatively intact but NO availability may become reduced due, for instance, to oxidative stress. In spastic sphincters, relaxation still takes place, but the pattern of contractions is affected, probably due to un-coordinated or disorganized signaling among the various tissues involved.

Sphincter dysfunction is considered primary when it is not associated with another systemic disease.

Sphincter dysfunction can also be secondary to other diseases. For instance, diabetes may result in damage to the nerves of the enteric nervous system, giving rise to diabetic sphincter dysfunction in the stomach, esophagus or the intestines. In systemic sclerosis, or other connective tissue diseases, for instance, smooth muscle is replaced by fibrotic tissue, making the muscles rigid and unable to relax.

Similarly, the role played by the enteric nervous system (ENS) in neurological or neurodegenerative disorders, as well as neuronal injury, has also become increasingly evident. Pathogenic mechanisms that give rise to CNS disorders might also lead to ENS dysfunction, and in particular sphincter dysfunction, and nerves that interconnect the ENS and CNS can be conduits for disease spread. ENS dysfunction has been shown in the etiopathogenesis of autism spectrum disorder, motor neuron disease (also referred to as amyotrophic lateral sclerosis; ALS), transmissible spongiform encephalopathies, Parkinson disease (PD) and Alzheimer disease (AD). Animal models suggest that common pathophysiological mechanisms account for the frequent gastrointestinal comorbidity in these conditions. Other neuronal, neurodegenerative diseases that are accompanied by a component of GI dysfunction are dementias, synucleinopathies, multiple system atrophy (MSA), Lewy body dementia, prion diseases, multiple sclerosis, frontotemporal lobar degeneration, Huntington's disease, and spinocerebellar ataxia (spinal muscular atrophy).

Dysfunction of the ENS, and in particular of the sphincters, may also develop as a result of cerebrovascular injury (such as stroke), brain surgery, and head or neck trauma.

Dysfunction of the ENS, and in particular of the sphincters, may also develop as a result of paraneoplastic syndromes—autoimmune diseases that attack neurons of the ENS and associated with different cancers, such as small cell lung cancer, breast or ovarian cancer, multiple myeloma and Hodgkin's lymphoma.

Nitrate-type NO donors, such as sublingual isosorbide dinitrate have been used as a treatment of achalasia. However, the effect of nitrates is of short duration. In addition, nitrates are known to possess limitations that preclude their long term use, such as the development of tolerance. This therapy rarely yields satisfactory long term relief.

There are also reports of the use of PDE5 inhibitors (e.g., sildenafil) for the treatment for achalasia. According to a report from 2000, sildenafil was able to reduce LES pressure but clinical symptoms were not improved. In addition, patients reported side effects such as dizziness and headaches.

### Methods of Treatment

As used herein, the terms “subject” and “patient” are used interchangeably to refer to an animal (e.g., a bird such as a chicken, quail or turkey, or a mammal), preferably a “mammal” including a non-primate (e.g., a cow, pig, horse, sheep, rabbit, guinea pig, rat, cat, dog, and mouse) and a primate (e.g., a monkey, chimpanzee and a human), and more preferably a human. In one embodiment, the subject is a non-human animal such as a farm animal (e.g., a horse, cow, pig or sheep), or a pet (e.g., a dog, cat, guinea pig or rabbit). In a preferred embodiment, the subject or patient is a human.

As used herein, the term a “patient in need thereof” is used to refer to a patient suffering from one of the gastrointestinal sphincter disorders here described, for example gastrointestinal sphincter dysfunction, spastic sphincters or hypertensive sphincters.

In some embodiments, the “patient in need thereof” is a patient with idiopathic achalasia or who has been diagnosed with achalasia or who is genetically predisposed to the development of achalasia. In still other embodiments a patient in need thereof is a person (usually a child, sometimes an infant) that has been genetically tested and found to have a mutation in a gene that predisposes him or her to the development of achalasia, even although he or she may not yet display or experience any signs or symptoms of achalasia. In some instances, a “patient in need thereof” experiences symptoms of achalasia even although a diagnosis may not yet have been made.

As used herein, the term “treat”, “treating” or “treatment” with regard to a disorder or disease refers to alleviating or abrogating the cause and/or effects or symptoms or clinical manifestations of the disorder or disease. As used herein, the terms “treat”, “treatment” and “treating” refer to the reduction or amelioration or slowing down of the progression, severity and/or duration of gastrointestinal sphincter dysfunction, for example, dysfunction of a sphincter of the gastrointestinal tract, a spastic sphincter of the gastrointestinal tract or a hypertensive sphincter of the gastrointestinal tract.

In some embodiments, the terms “treat”, “treatment” and “treating” refer or the reduction, amelioration or slowing down of the progression, the severity and/or the duration of one or more symptoms or clinical manifestations (preferably, one or more measurable symptoms or clinical manifestations) of the condition, as a result of the administration of one or more therapies (e.g., an NEP inhibitor or a pharmaceutically acceptable salt thereof, either alone or in combination).

In some embodiments, the terms “treat,” “treatment” and “treating” refer to delaying the onset of a symptom or set of symptoms or clinical manifestations or to delaying the onset of a loss in certain physical function (e.g., ability of the LES or another gastrointestinal sphincter to relax).

In some embodiments, the terms “treat,” “treatment” and “treating” refer to the amelioration of at least one measurable physical manifestation of dysfunction of a gastrointestinal tract sphincter such as achalasia (e.g., aperistalsis). In other embodiments the terms “treat”, “treatment” and “treating” refer to the reduction, inhibition or slowing down of the progression of said condition, either physically by, e.g., stabilization of a measurable symptom or set of symptoms (e.g., dysphagia or pain), or physiologically by, e.g., stabilization of a measurable parameter (increased resting pressure of the LES or another sphincter assessed manometrically), or both. As used herein, the term “treating”, “treat” or “treatment” also refers to averting the cause and/or effects or clinical manifestation of a disease or disorder or one of the symptoms developed as a result of the disease or disorder prior to the disease or disorder fully manifesting itself.

“Treatment” can involve administering a compound described herein to a patient diagnosed with a gastrointestinal sphincter dysfunction here described and may involve administering the compound to a patient who does not have active symptoms. Conversely, treatment may involve administering the compositions to a patient at risk of developing a particular disease, or to a patient reporting one or more of the symptoms of a disease, even though a diagnosis of this disease may not have been made.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. The therapeutically effective amount of the compound to be administered will be governed by such considerations, and is the minimum amount necessary to ameliorate, cure or treat the disease or disorder or one or more of its symptoms.

The term “prophylactically effective amount” refers to an amount effective in preventing or substantially lessening the chances of acquiring a disorder or in reducing the severity of the disorder or one or more of its symptoms before it is acquired or before the symptoms fully develop.

In one aspect, the invention provides a method of treating achalasia, comprising administering a therapeutically or prophylactically effective amount of an NEP inhibitor, or pharmaceutically acceptable salt thereof, alone or in combination with a therapeutically or prophylactically effective amount of one or more additional therapeutic agents to a patient in need thereof patient.

In a further aspect, the invention provides a use of an NEP inhibitor or a pharmaceutically acceptable salt thereof, in the manufacture of a medicament for the treatment of achalasia in a patient in need thereof.

In another aspect, the invention provides pharmaceutical compositions comprising a NEP inhibitor or a pharmaceutically acceptable salt thereof, for use in the treatment of achalasia in a patient in need thereof. In another aspect, the invention provides pharmaceutical compositions comprising an NEP inhibitor, or a pharmaceutically acceptable salt thereof, in combination with one or more additional therapeutic agents, for use in the treatment of achalasia in a patient in need thereof.

In still a further aspect, the invention provides a kit comprising at least two separate unit dosage forms (A) and (B), wherein (A) is a therapeutic agent, a combination of more than one therapeutic agent, a pharmaceutically acceptable salt thereof, or a pharmaceutical composition thereof, and (B) is an NEP inhibitor, a pharmaceutically acceptable salt thereof, or a pharmaceutical composition comprising an NEP inhibitor or a pharmaceutically acceptable salt thereof for use in the treatment of achalasia in a patient in need thereof.

In some embodiments of the above methods, uses, compositions and kits, the patient in need thereof is an adult. In other embodiments the patient is a child. In still other embodiments the patient in need thereof is an infant.

In some embodiments of the above methods, uses, compositions and kits, the administration of an NEP inhibitor or pharmaceutically acceptable salt thereof, alone or in combination with another therapeutic agent, results in an observable or measurable decrease in the degree of failure of the lower esophageal sphincter to relax with and immediately after swallowing. In other embodiments, it results in an observable or measurable decrease in the degree of failure of the LES to relax with or immediately after swallowing. In other embodiments, it results in an observable or measurable decrease in the degree of aperistalsis of the esophageal body in response to swallowing. In other embodiments, it results in an observable or measurable decrease in the degree of dysphagia. In other embodiments, it results in an observable or measurable reduction in regurgitation of undigested food. In still other embodiments, it results in an observable or measurable decrease in the progression of disease. In other embodiments, it results in an observable or measurable reduction in inflammation around the myenteric plexus.

In some embodiments of the above methods, uses, compositions and kits, the administration of an NEP inhibitor or pharmaceutically acceptable salt thereof, alone or in combination with another therapeutic agent, results in an observable or measurable reduction in heartburn. In other embodiments, it results in a measurable or observable reduction in chest pain. In other embodiments, it results in an observable or measurable reduction of wheezing. In other embodiments, it results in an observable or measurable reduction of coughing. In other embodiments, it results in an observable or measurable reduction of hoarseness. In other embodiments, it results in an observable or measurable reduction of sore throat. In other embodiments, it results in an observable or measurable reduction of coughing when lying in a horizontal position. In other embodiments, it results in an observable or measurable reduction in the degree of retention of food in the esophagus. In other embodiments, it results in an observable or measurable reduction of aspiration of food into the lungs. In other embodiments, it results in an observable or measurable reduction of cardiospasm.

In some embodiments of the above methods, uses, compositions and kits, the administration of an NEP inhibitor or pharmaceutically acceptable salt thereof, alone or in combination with another therapeutic agent, results in an observable or measurable inhibition of weight loss.

In some embodiments of the above methods, uses, compositions and kits, the administration of an NEP inhibitor or a pharmaceutically acceptable salt thereof, alone or in combination with another therapeutic agent, results in an observable or measurable improvement in the ability of the LES to relax with and immediately after swallowing. In other embodiments, it results in an observable or measurable improvement in peristalsis of the esophagus.

In other embodiments, it results in an observable or measurable improvement in the ability to swallow liquids or solids. In other embodiments, it results in an observable or measurable improvement in chest pain. In still other embodiments, it results in an observable or measurable improvement in heartburn.

In some embodiments of the above methods, uses, compositions and kits, the administration of an NEP inhibitor or a pharmaceutically acceptable salt thereof, alone or in combination with another therapeutic agent, results in a measurable reduction in the LES pressure with and immediately after swallowing as measured by manometry.

In some embodiments of the above methods, uses, compositions and kits, the administration of an NEP inhibitor or a pharmaceutically acceptable salt thereof, alone or in combination with another therapeutic agent, results in a measurable increase in the percentage of relaxation of the LES with and immediately after swallowing as measured by manometry.

In some embodiments of the above methods, uses, compositions and kits, the administration of an NEP inhibitor or a pharmaceutically acceptable salt thereof, alone or in combination with another therapeutic agent, results in a measurable decrease in LES compared to intragastric pressure after swallowing as measured by manometry.

In some embodiments of the above methods, uses, compositions and kits, the administration of an NEP inhibitor, or a pharmaceutically acceptable salt thereof, or a pharmaceutical composition comprising an NEP inhibitor or a pharmaceutically acceptable salt thereof, alone or in combination with another therapeutic agent, results in the improvement or reduction, or slowing down in the development of one or more symptoms selected from: dysphagia, regurgitation of undigested food, chest pain, cardiospasm, heartburn, shortness of breath, wheezing, cough, coughing when lying in a horizontal position, retention of food in the esophagus, aspiration of food into the lungs, vomiting, projectile vomiting, bloating, fullness, nausea.

In some embodiments of the above methods, uses, compositions and kits, the administration of an NEP inhibitor or a pharmaceutically acceptable salt thereof, or a pharmaceutical composition comprising an NEP inhibitor or a pharmaceutically acceptable salt thereof, alone or in combination with another therapeutic agent, to a patient in need thereof, is aimed at treating one or more symptoms selected from: dysphagia, regurgitation of undigested food, chest pain, heartburn, shortness of breath, wheezing, cough, coughing when lying in a horizontal position, retention of food in the esophagus, aspiration of food into the lungs, vomiting, projectile vomiting, bloating, fullness, nausea.

The pyloric valve is a sphincter-type valve that controls the opening between the bottom end of the stomach and the beginning of the small intestine.

The pyloric valve's principal function is to control the flow of partially digested material from the stomach into the duodenum, the first part of the small intestine, where most of the nutrients get extracted from what is eaten. When the valve is working well, it opens slightly a few times a minute to allow a small amount of food to move into the duodenum. Its secondary function is to prevent bile from flowing back from the small intestine into the stomach (bile reflux).

When the pyloric valve is malfunctioning, it creates discomfort and many serious medical problems.

When the pylorus completely fails to open (in the absence of a physical obstruction to the pylorus), the most common symptom is projectile or severe vomiting, accompanied by distension of the stomach and pain, as partially undigested food accumulates and is unable to pass into the intestines. This occurs, for example, in non-obstructive pyloric stenosis and congenital hypertrophic pyloric stenosis. The latter may be familial or idiopathic.

The ileocecal valve (ICV) is a sphincter located at the junction of the end of the small intestine and beginning of the large intestine. Its purpose is to prevent bacteria laden material in the large intestine from ‘back flowing’ into the small intestine and contaminating it.

Once material has been allowed to pass through the ileocecal valve to enter the large intestine, the valve closes again to prevent back flow from the large intestine. One complication of an incompetent ICV would be small intestinal bacterial overgrowth (SIBO).

The sphincter of Oddi is a muscular valve that controls the flow of digestive juices (bile and pancreatic juice) through ducts from the liver and pancreas into the first part of the small intestine (duodenum). Sphincter of Oddi dysfunction (SOD) describes the situation when the sphincter does not relax at the appropriate time (due to spasm). The back-up of juices causes episodes of severe abdominal pain.

Patients with a similar pain problem, but who have little or no abnormalities on blood tests and standard scans (including MRCP), are categorized as having SOD Type III. The episodes of pain are assumed due to intermittent spasm of the sphincter. It is very difficult to effectively evaluate and manage patients with Type III SOD.

Hirschsprung's disease (HD) is a form of megacolon that occurs when part or all of the large intestine have no ganglion cells and therefore cannot function. During normal prenatal development, cells from the neural crest migrate into the large intestine (colon) to form the networks of nerves called the myenteric plexus (Auerbach plexus) (between the smooth muscle layers of the gastrointestinal tract wall) and the submucosal plexus (Meissner plexus) (within the submucosa of the gastrointestinal tract wall). In Hirschsprung's disease, the migration is not complete and part of the colon lacks these nerve bodies that regulate the activity of the colon. The affected segment of the colon cannot contract and pass stool through the colon, creating an obstruction. In most affected people, the disorder affects the part of the colon that is nearest the anus, i.e., the anal sphincters and related area. In rare cases, the lack of nerve bodies involves more of the colon. In five percent of cases, the entire colon is affected. The stomach and esophagus may be affected too.

Hirschsprung's disease occurs in about one in 5,000 of live births. It is usually diagnosed in children, and affects boys more often than girls. About 10% of cases are familial.

Typically, Hirschsprung's disease is diagnosed shortly after birth, although diagnosis may rarely be delayed well into adulthood. It is usually diagnosed because of the presence of megacolon, or because the baby fails to pass the first stool (meconium) within 48 hours of delivery. Normally, 90% of babies pass their first meconium within 24 hours, and 99% within 48 hours. Other features may include green or brown vomit, explosive diarrhea after a doctor inserts a finger into the rectum, swelling of the abdomen, and intermittent passage of excessive gas and diarrhea.

Some cases are diagnosed later, into childhood, but usually before age 10. The child may experience fecal retention, constipation, and/or abdominal distention. With an incidence of one in 5,000 births, the most cited feature is absence of ganglion cells: notably in males, 75 percent have none in the end of the colon (recto-sigmoid) and eight percent lack ganglion cells in the entire colon. The enlarged section of the bowel is found proximal to the narrowed, aganglionic section, closer to the end of the bowel, in the sphincter area. The absence of ganglion cells results in a persistent over-stimulation of nerves in the affected region, resulting in contraction.

The lack of ganglion cells in the myenteric and submucosal plexus is well-documented in Hirschsprung's disease. The segment lacking neurons (aganglionic) becomes constricted, causing the normal, proximal section of bowel to become distended with feces. Definitive diagnosis is made by suction biopsy of the distally narrowed segment. A histologic examination of the tissue would show a lack of ganglionic nerve cells. Diagnostic techniques involve anorectal manometry, barium enema, and rectal biopsy. The suction rectal biopsy is considered the current international gold standard in the diagnosis of Hirschsprung's disease.

Radiologic findings may also assist with diagnosis. Cineanography (fluoroscopy of contrast medium passing anorectal region) assists in determining the extent of colon involvement. There are a variety of surgical treatments for Hirschsprung's disease, generally involving removal (resection) of the abnormal section of the colon, followed by re-anastomosis.

### NEP Inhibitors

For purposes of this disclosure, the chemical elements are identified in accordance with the Periodic Table of the Elements, CAS version, and the Handbook of Chemistry and Physics, 75^{th} Ed. 1994. Additionally, general principles of organic chemistry are described in “Organic Chemistry”, Thomas Sorrell, University Science Books, Sausalito: 1999, and “March's Advanced Organic Chemistry”, 5^{th} Ed., Smith, M. B. and March, J., eds. John Wiley & Sons, New York: 2001, which are herein incorporated by reference in their entirety.

NEP inhibitors that can be utilized in the methods, uses and compositions of this invention include, but are not limited to, sacubitril. In other embodiments, the NEP inhibitor is racecadotril, a prodrug or its active metabolite thiorphan. In other embodiments, the NEP inhibitor is ecadotril. In other embodiments, the NEP inhibitor is TD-1439, TD-0714 or TD-0212 (Theravance compounds currently in clinical trials). In still other embodiments, the NEP inhibitor is selected from daglutril, ilepatril, SLV-338, UK-447841, VPN-489, LBQ657 or LHW-090.

Sacubitril is an inhibitor of the NEP enzyme that proteolytically degrades the natriuretic peptides atrial and brain natriuretic peptides (ANP and BNP, respectively) and thus elevates the plasma levels of these peptides. Elevated levels of natriuretic peptides lead to elevated levels of cGMP through activation of the GC-A receptor. Sacubitril is a component of the combination drug sacubitril-valsartan, known during clinical trials as LCZ696 and marketed in the US as Entresto. It is approved in the United States, Europe, and several other regions for the treatment of heart failure.

An NEP inhibitor alone, rather than in combination with an angiotensin receptor blocker (ARB, as is the case in the approved product Entresto) is the preferred drug. Valsartan (an ARB currently approved in combination with sacubitril for heart failure) would further lower blood pressure, which is already a complication in patients with acute respiratory distress syndrome (ARDS) or sepsis. Furthermore, in animal models, both angiotensin converting enzyme (ACE) inhibitors and ARBs upregulate angiotensin converting enzyme 2 (ACE2) receptor expression in the heart.

Treatment with Entresto leads to increases in endogenous plasma levels of natriuretic peptides (ANP and BNP) and (plasma and urinary) cGWIP in patients (Entresto prescribing information; McMurray, J. J. V., et al. “Angiotensin-Neprilysin Inhibition versus Enalapril in Heart Failure” AT Engl, I Med, 371, 993-1004, 2014).

Currently there are a number of other pharmacologic agents in clinical use that increase signaling through the GC-A—cGiVIP pathway. In one embodiment, the agents that can be utilized in the methods, uses and compositions of the invention are GC-A agonists. In some embodiments the GC-A agonist is ANP. In other embodiments, it is BNP. In still another embodiment, the agent is an ANP or BNP analogue.

There exist different forms of BNP that may be utilized in the methods, uses and compositions of the invention including, but not limited to, nesiritide.

### Pharmaceutically Acceptable Salts

In some embodiments of the methods, uses, pharmaceutical compositions and kits, the NEP inhibitor may be provided as (i) the compound itself (e.g., as the free base); (ii) a pharmaceutically acceptable salt of the compound; or (iii) part of a pharmaceutical composition. In some embodiments of the above methods, uses, pharmaceutical compositions and kits, the additional therapeutic agent may be provided as (i) the compound itself (e.g., as the free base); (ii) a pharmaceutically acceptable salt of the compound; (iii) or part of a pharmaceutical composition.

The phrase “pharmaceutically acceptable salt,” as used herein, refers to pharmaceutically acceptable organic or inorganic salts of a compound described herein. For use in medicine, the salts of the compounds described herein will be pharmaceutically acceptable salts. Other salts may, however, be useful in the preparation of the compounds described herein or of their pharmaceutically acceptable salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

Pharmaceutically acceptable salts of the compounds described herein include those derived from suitable inorganic and organic acids and bases. In some embodiments, the salts can be prepared in situ during the final isolation and purification of the compounds. In other embodiments the salts can be prepared from the free form of the compound in a separate synthetic step.

When the compound described herein is acidic or contains a sufficiently acidic bioisostere, suitable “pharmaceutically acceptable salts” refers to salts prepared form pharmaceutically acceptable non-toxic bases including inorganic bases and organic bases. Salts derived from inorganic bases include aluminum, ammonium, calcium, copper, ferric, ferrous, lithium, magnesium, manganic salts, manganous, potassium, sodium, zinc and the like. Particular embodiments include ammonium, calcium, magnesium, potassium and sodium salts. Salts derived from pharmaceutically acceptable organic non-toxic bases include salts of primary, secondary and tertiary amines, substituted amines including naturally occurring substituted amines, cyclic amines and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine tripropylamine, tromethamine and the like.

When the compound described herein is basic or contains a sufficiently basic bioisostere, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particular embodiments include citric, hydrobromic, hydrochloric, maleic, phosphoric, sulfuric and tartaric acids. Other exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methane sulfonate, ethanesulfonate, benzenesulfonate, p-toluenesulfonate, and pamoate (i.e., 1,1′-methylene-bis-(2-hydroxy-3-naphthoate)) salts.

The preparation of the pharmaceutically acceptable salts described above and other typical pharmaceutically acceptable salts is more fully described by Berg et al., “Pharmaceutical Salts,” J. Pharm. Sci., 1977:66: 1-19, incorporated herein by reference in its entirety. Compounds, compositions and kits of the invention are also useful for veterinary treatment of companion animals, exotic animals and farm animals, including, without limitation, dogs, cats, mice, rats, hamsters, gerbils, guinea pigs, rabbits, horses, pigs and cattle.

### Methods of Administration and Co-Administration

In some embodiments of the above methods and uses, the NEP inhibitor is administered before a symptom of achalasia fully develops in said patient. In other embodiments of the above methods and uses, the NEP inhibitor is administered after one or more symptoms of achalasia develops in said patient.

As used herein, the terms “in combination” or “co-administration” can be used interchangeably to refer to the use of more than one therapy (e.g., an NEP inhibitor and one or more additional therapeutic agents). The use of the terms does not restrict the order in which therapies (e.g., the NEP inhibitor and the additional therapeutic agents) are administered to a subject.

In some embodiments, the NEP inhibitor is administered prior to, at the same time or after the initiation of treatment with another therapeutic agent.

In some embodiments of the above methods and uses, the additional therapeutic agent and the NEP inhibitor are administered simultaneously. In other embodiments of the above methods and uses, the additional therapeutic agent and the NEP inhibitor are administered sequentially or separately.

In some embodiments, the above pharmaceutical compositions or kits comprise (a) an NEP inhibitor as discussed above or a pharmaceutically acceptable salt thereof, and (b) a pharmaceutically acceptable carrier, vehicle or adjuvant. In some embodiments, the pharmaceutical composition or kit comprises (a) one or more additional therapeutic agents as discussed above, or a pharmaceutically acceptable salt thereof, and (b) a pharmaceutically acceptable carrier, vehicle or adjuvant. In some embodiments, the pharmaceutical composition comprises (i) an NEP inhibitor as discussed above, or a pharmaceutically acceptable salt thereof, (ii) one or more additional therapeutic agents as discussed above, or a pharmaceutically acceptable salt thereof, and (iii) a pharmaceutically acceptable carrier, vehicle or adjuvant.

The NEP inhibitor and pharmaceutical compositions described herein can be used in combination therapy with one or more additional therapeutic agents. For combination treatment with more than one active agent, the additional active agents may be in the same dosage form or in separate dosage forms. Wherein the additional active agents are present in separate dosage forms, the active agents may be administered separately or in conjunction with the NEP inhibitor. In addition, the administration of one agent may be prior to, concurrent to, or subsequent to the administration of the other agent.

When co-administered with other agents, e.g., when co-administered with another agent, an “effective amount” of the second agent will depend on the type of drug used. Suitable dosages are known for approved agents and can be adjusted by the skilled artisan according to the condition of the subject, the type of condition(s) being treated and the amount of a compound described herein being used. In cases where no amount is expressly noted, an effective amount should be assumed. For example, compounds described herein can be administered to a subject in a dosage range from between about 0.001 to about 100 mg/kg body weight/day, from about 0.001 to about 50 mg/kg body weight/day, from about 0.001 to about 30 mg/kg body weight/day, from about 0.001 to about 10 mg/kg body weight/day.

When “combination therapy” is employed, an effective amount can be achieved using a first amount of an NEP inhibitor or a pharmaceutically acceptable salt thereof and a second amount of an additional suitable therapeutic agent (e.g., another NEP inhibitor, a PDE inhibitor, arginine, a NO modulator, a cGMP modulator, a therapeutic that increases the function of nitric oxide synthase, etc.).

In one embodiment of this invention, the NEP inhibitor and the additional therapeutic agent are each administered in an effective amount (i.e., each in an amount which would be therapeutically effective if administered alone). In another embodiment, the NEP inhibitor and the additional therapeutic agent are each administered in an amount which alone does not provide a therapeutic effect (“a sub-therapeutic dose”). In yet another embodiment, the NEP inhibitor can be administered in an effective amount, while the additional therapeutic agent is administered in a sub-therapeutic dose. In still another embodiment, the NEP inhibitor can be administered in a subtherapeutic dose, while the additional therapeutic agent, for example, a suitable anti-inflammatory agent is administered in an effective amount.

“Co-administration” encompasses administration of the first and second amounts of the compounds in an essentially simultaneous manner, such as in a single pharmaceutical composition, for example, capsule or tablet having a fixed ratio of first and second amounts, or in multiple, separate capsules or tablets for each. In addition, co-administration also encompasses use of each compound in a sequential manner in either order. When co-administration involves the separate administration of the first amount of an NEP inhibitor and a second amount of an additional therapeutic agent, the compounds are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life and kinetic profile. For example, an NEP inhibitor and the second therapeutic agent can be administered in any order within about 24 hours of each other, within about 16 hours of each other, within about 8 hours of each other, within about 4 hours of each other, within about 1 hour of each other or within about 30 minutes of each other, within about 5 minutes of each other, etc.

More, specifically, a first therapy (e.g., a prophylactic or therapeutically used NEP inhibitor) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks prior to), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks subsequent to) the administration of a second therapy (e.g., an additional therapeutic agent or prophylactic agent described herein) to a subject.

### Combination Therapies

In some embodiments, of the above methods, uses, compositions and kits, the additional therapeutic agent or agents can be a compound that inhibits the degradation of cGMP. Suitable compounds that inhibit the degradation of cGMP include, but are not limited to PDE5 inhibitors and PDE9 inhibitors.

PDE5 inhibitors, include, for example, sildenafil (Viagra©) and related agents such as avanafil, lodenafil, mirodenafil, sildenafil citrate (Revatio®), tadalafil (Cialis© or Adcirca®), vardenafil (Levitra©) and udenafil; alprostadil; dipyridamole and PF-00489791; and

PDE9 inhibitors, include for example, PF-04447943.

In some embodiments of the above methods, uses, compositions and kits, the additional therapeutic agent or agents may be selected from one or more of the following:

(1) Endothelium-derived releasing factor (EDRF) or NO gas.

(2) NO donors such as a nitrosothiol, a nitrite, a sydnonimine, a NONOate, a N-nitrosamine, a N-hydroxyl nitrosamine, a nitrosimine, nitrotyrosine, a diazetine dioxide, an oxatriazole 5-imine, an oxime, a hydroxylamine, a N-hydroxyguanidine, a hydroxyurea or a furoxan. Some examples of these types of compounds include: glyceryl trinitrate (also known as GTN, nitroglycerin, nitroglycerine, and trinitrogylcerin), the nitrate ester of glycerol; sodium nitroprusside (SNP), wherein a molecule of nitric oxide is coordinated to iron metal forming a square bipyramidal complex; 3-morpholinosydnonimine (SIN-1), a zwitterionic compound formed by combination of a morpholine and a sydnonimine; S-nitroso-N-acetylpenicillamine (SNAP), an N-acetylated amino acid derivative with a nitrosothiol functional group; diethylenetriamine/NO (DETA/NO), a compound of nitric oxide covalently linked to diethylenetriamine; an m-nitroxymethyl phenyl ester of acetyl salicylic acid. More specific examples of some of these classes of NO donors include: the classic nitrovasodilators, such as organic nitrate and nitrite esters, including nitroglycerin, amyl nitrite, isosorbide dinitrate, isosorbide 5-mononitrate, and nicorandil; isosorbide (Dilatrate®-SR, Imdur®, Ismo®, Isordil®, Isordil®, Titradose®, Monoket®), 3-morpholinosydnonimine; linsidomine chlorohydrate (“SIN-1”); S-nitroso-N-acetylpenicillamine (“SNAP”); S-nitrosoglutathione (GSNO), sodium nitroprusside, S-nitrosoglutathione mono-ethyl-ester (GSNO-ester), 6-(2-hydroxy-1-methyl-nitrosohydrazino)-N-methyl-1-hexanamine or diethylamine NONOate.

(3) Other substances that enhance cGMP concentrations such as protoporphyrin IX, arachidonic acid and phenyl hydrazine derivatives.

(4) Nitric Oxide Synthase substrates: for example, n-hydroxyguanidine based analogs, such as N[G]-hydroxy-L-arginine (NOHA), 1-(3,4-dimethoxy-2-chlorobenzylideneamino)-3-hydroxyguanidine, and PR5 (1-(3,4-dimethoxy-2-chlorobenzylideneamino)-3-hydroxyguanidine); L-arginine derivatives (such as homo-Arg, homo-NOHA, N-tert-butyloxy- and N-(3-methyl-2-butenyl)oxy-L-arginine, canavanine, epsilon guanidine-carpoic acid, agmatine, hydroxyl-agmatine, and L-tyrosyl-L-arginine); N-alkyl-N′-hydroxyguanidines (such as N-cyclopropyl-N′-hydroxyguanidine and N-butyl-N′-hydroxyguanidine), N-aryl-N′-hydroxyguanidines (such as N-phenyl-N′-hydroxyguanidine and its para-substituted derivatives which bear—F, —CI, -methyl, —OH substituents, respectively); guanidine derivatives such as 3-(trifluoromethyl) propylguanidine.

(5) Compounds which enhance eNOS transcription.

(6) NO independent heme-independent sGC activators, including, but not limited to:
BAY 58-2667 (described in patent publication DE19943635)

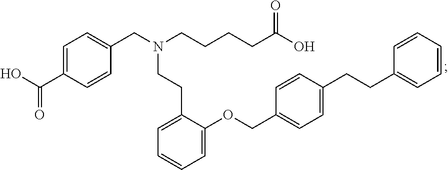 [SMILES: O=C(O)CCCCN(CCc1ccccc1OCc1ccc(CCc2ccccc2)cc1)Cc1ccc(C(=O)O)cc1]

HMR-1766 (ataciguat sodium, described in patent publication WO2000002851)

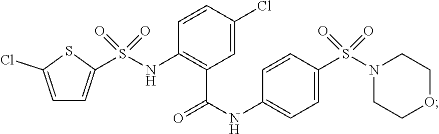 [SMILES: O=C(Nc1ccc(S(=O)(=O)N2CCOCC2)cc1)c1cc(Cl)ccc1NS(=O)(=O)c1ccc(Cl)s1]

S 3448 (2-(4-chloro-phenylsulfonylamino)-4,5-dimethoxy-N-(4-(thiomorpholine-4-sulfonyl)-phenyl)-benzamide (described in patent publications DE19830430 and WO2000002851)

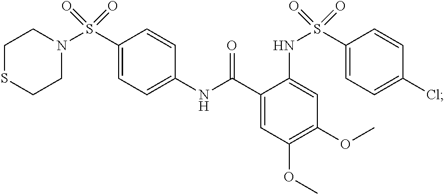 [SMILES: COc1cc(NS(=O)(=O)c2ccc(Cl)cc2)c(C(=O)Nc2ccc(S(=O)(=O)N3CCSCC3)cc2)cc1OC]

(7) Heme-dependent, NO-independent sGC stimulators including, but not limited to:
YC-1 (see patent publications EP667345 and DE19744026)

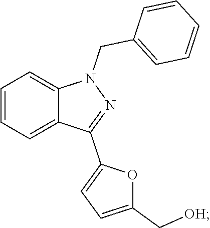 [SMILES: OCc1ccc(-c2nn(Cc3ccccc3)c3ccccc23)o1]

riociguat (BAY 63-2521, Adempas®, described in DE19834044)

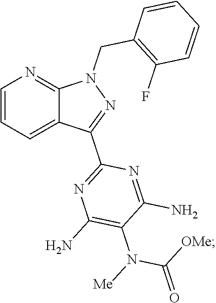 [SMILES: COC(=O)N(C)c1c(N)nc(-c2nn(Cc3ccccc3F)c3ncccc23)nc1N]

neliciguat (BAY 60-4552, described in WO 2003095451)

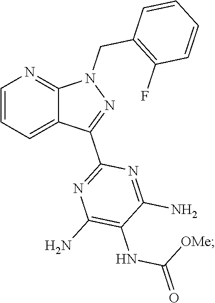 [SMILES: COC(=O)Nc1c(N)nc(-c2nn(Cc3ccccc3F)c3ncccc23)nc1N]

vericiguat (BAY 1021189)

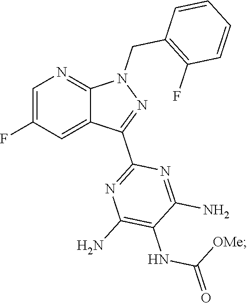 [SMILES: COC(=O)Nc1c(N)nc(-c2nn(Cc3ccccc3F)c3ncc(F)cc23)nc1N]

BAY 41-2272 (described in DEI 9834047 and DEI 9942809)

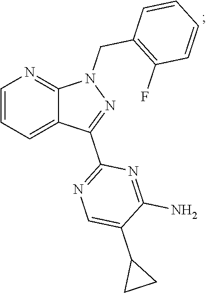 [SMILES: Nc1nc(-c2nn(Cc3ccccc3F)c3ncccc23)ncc1C1CC1]

BAY 41-8543 (described in DE19834044)

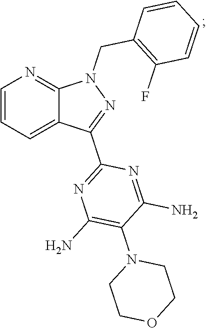 [SMILES: Nc1nc(-c2nn(Cc3ccccc3F)c3ncccc23)nc(N)c1N1CCOCC1]

etriciguat (described in WO 2003086407)

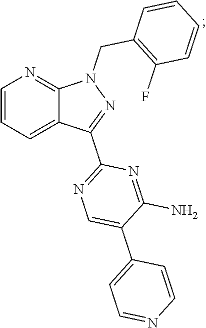 [SMILES: Nc1nc(-c2nn(Cc3ccccc3F)c3ncccc23)ncc1-c1ccncc1]

CFM-1571 (described in patent publication WO2000027394)

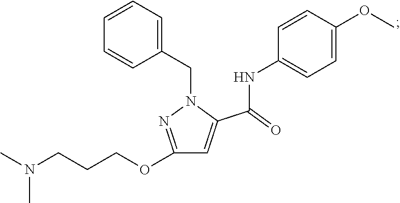

(8) A-344905, its acrylamide analogue A-350619 and the aminopyrimidine analogue A-778935

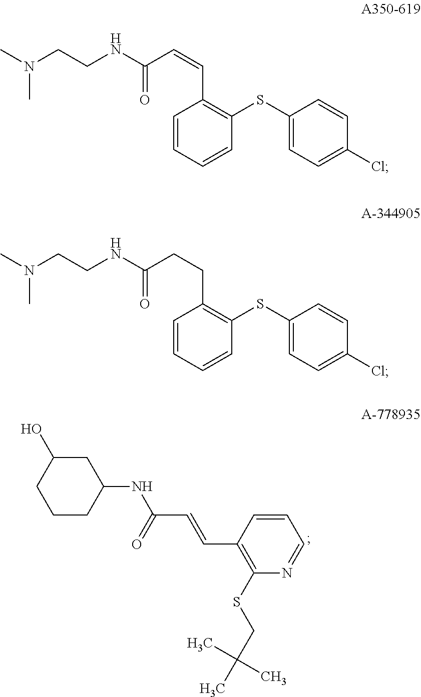 [SMILES: CN(C)CCNC(=O)/C=C\c1ccccc1Sc1ccc(Cl)cc1 ; CN(C)CCNC(=O)CCc1ccccc1Sc1ccc(Cl)cc1 ; CC(C)(C)CSc1ncccc1/C=C/C(=O)NC1CCCC(O)C1]

and other sGC stimulators described in one of publications US20090209556, U.S. Pat. No. 8,455,638, US20110118282 (WO2009032249), US20100292192, US20110201621, U.S. Pat. Nos. 7,947,664, 8,053,455 (WO2009094242), US20100216764, U.S. Pat. No. 8,507,512, (WO2010099054) US20110218202 (WO2010065275), US20130012511 (WO2011119518), US20130072492 (WO2011149921), US20130210798 (WO2012058132) and other compounds described in Tetrahedron Letters (2003), 44(48): 8661-8663.

(9) Calcium channel blockers of the following types: dihydropyridine calcium channel blockers such asamlodipine (Norvasc®), aranidipine (Sapresta®), azelnidipine (Calblock®), barnidipine (HypoCa®), benidipine (Coniel®), cilnidipine (Atelec®, Cinalong®, Siscard®), clevidipine (Cleviprex®), diltiazem, efonidipine (Landel®), felodipine (Plendil®), lacidipine (Motens®, Lacipil®), lercanidipine (Zanidip®), manidipine (Calslot®, Madipine®), nicardipine (Cardene®, Carden SR®), nifedipine (Procardia®, Adalat®), nilvadipine (Nivadil®), nimodipine (Nimotop®), nisoldipine (Baymycard®, Sular®, Syscor®), nitrendipine (Cardif®, Nitrepin®, Baylotensin®), pranidipine (Acalas®), isradipine (Lomir®); phenylalkylamine calcium channel blockers such as verapamil (Calan®, Isoptin®)

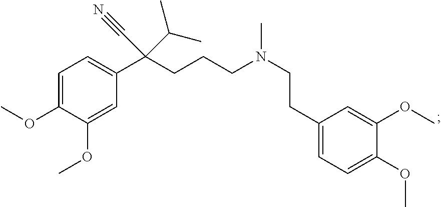

and gallopamil (Procorum®, D600);
benzodiazepines such asdiltiazem (Cardizem®)

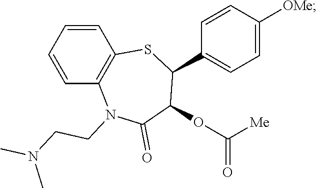 [SMILES: COc1ccc([C@@H]2Sc3ccccc3N(CCN(C)C)C(=O)[C@@H]2OC(C)=O)cc1]

and nonselective calcium channel inhibitors such as mibefradil, bepridil, fluspirilene, and fendiline.

(10) Endothelin receptor antagonists (ERAs) such as the dual (ETA and ETB) endothelin receptor antagonist bosentan (Tracleer®), sitaxentan (Thelin®) or ambrisentan (Letairis®).

(11) Prostacyclin derivatives or analogues, such asprostacyclin (prostaglandin I2), epoprostenol (synthetic prostacyclin, Flolan®), treprostinil (Remodulin®), iloprost (Ilomedin®), iloprost (Ventavis®); and oral and inhaled forms of Remodulin® under development.

(12) Antihyperlipidemics such as the following types: bile acid sequestrants like cholestyramine, colestipol, colestilan, colesevelam or sevelamer; statins like atorvastatin, simvastatin, lovastatin, fluvastatin, pitavastatin, rosuvastatin and pravastatin;
cholesterol absorption inhibitors such as ezetimibe;
other lipid lowering agents such as icosapent ethyl ester, omega-3-acid ethyl esters, reducol;
fibric acid derivatives such as clofibrate, bezafibrate, clinofibrate, gemfibrozil, ronifibrate, binifibrate, fenofibrate, ciprofibrate, choline fenofibrate;
nicotinic acid derivatives such as acipimox and niacin;
combinations of statins, niacin and intestinal cholesterol absorption-inhibiting supplements (ezetimibe and others) and fibrates; and
antiplatelet therapies such as clopidogrel bisulfate.

(13) Anticoagulants, such as the following types:
coumarines (Vitamin K antagonists) such as warfarin (Coumadin®), cenocoumarol, phenprocoumon and phenindione;
heparin and derivatives such as low molecular weight heparin, fondaparinux and idraparinux;
direct thrombin inhibitors such as argatroban, lepirudin, bivalirudin, dabigatran and ximelagatran (Exanta®); and
tissue-plasminogen activators, used to dissolve clots and unblock arteries, such as alteplase.

(14) Antiplatelet drugs such as, for instance, topidogrel, ticlopidine, dipyridamoleand aspirin.

(15) ACE inhibitors, for example the following types:
sulfhydryl-containing agents such as captopril (Capoten®) and zofenopril;
dicarboxylate-containing agents such as enalapril (Vasotec/Renitec®), Ramipril (Altace®/Tritace®/Ramace®/Ramiwin®), quinapril (Accupril®), perindopril (Coversyl®/Aceon®), lisinopril (Lisodur®/Lopril®/Novatec®/Prinivil®/Zestril®) and benazepril (Lotensin®);
phosphonate-containing agents such as fosinopril;
naturally occurring ACE inhibitors such as casokinins and lactokinins, which are breakdown products of casein and whey that occur naturally after ingestion of milk products, especially cultured milk;
the lactotripeptides Val-Pro-Pro and Ile-Pro-Pro produced by the probiotic Lactobacillus helveticus or derived from casein also having ACE-inhibiting and antihypertensive functions; other ACE inhibitors such as alacepril, delapril, cilazapril, imidapril, trandolapril, temocapril, moexipril and pirapril.

(16) Supplemental oxygen therapy.

(17) Beta blockers, such as the following types:
non-selective agents such as alprenolol, bucindolol, carteolol, carvedilol, labetalol, nadolol, penbutolol, pindolol, oxprenonol, acebutolol, sotalol, mepindolol, celiprolol, arotinolol, tertatolol, amosulalol, nipradilol, propranolol and timolol;
pt-Selective agents such as cebutolol, atenolol, betaxolol, bisoprolol, celiprolol, dobutamine hydrochloride, irsogladine maleate, carvedilol, talinolol, esmolol, metoprolol and nebivolol; and
P2-Selective agents such as butaxamine.

(18) Antiarrhythmic agents such as the following types:
Type I (sodium channel blockers) such as quinidine, lidocaine, phenytoin, propafenone;
Type III (potassium channel blockers) such as amiodarone, dofetilide and sotalol; and
Type V such as adenosine and digoxin.

(19) Diuretics such as thiazide diuretics, for example chlorothiazide, chlorthalidone and hydrochlorothiazide, bendroflumethiazide, cyclopenthiazide, methyclothiazide, polythiazide, quinethazone, xipamide, metolazone, indapamide, cicletanine; loop diuretics, such as furosemide and toresamide; potassium-sparing diuretics such as amiloride, spironolactone, canrenoate potassium, eplerenone and triamterene; combinations of these agents; other diuretics such as acetazolamid and carperitide.

(20) Direct-acting vasodilators such as hydralazine hydrochloride, diazoxide, sodium nitroprusside, cadralazine; other vasodilators such as isosorbide dinitrate and isosorbide 5-mononitrate.

(21) Exogenous vasodilators such as Adenocard® and alpha blockers.

(22) Alpha-1-adrenoceptor antagonists such as prazosin, indoramin, urapidil, bunazosin, terazosin and doxazosin; atrial natriuretic peptide (ANP), ethanol, histamine-inducers, tetrahydrocannabinol (THC) and papaverine.

(23) Bronchodilators of the following types:
short acting β2 agonists, such as albutamol or albuterol (Ventolin®) and terbutaline;
long acting β2 agonists (LABAs) such as salmeterol and formoterol;
anticholinergics such as pratropium and tiotropium; and
theophylline, a bronchodilator and phosphodiesterase inhibitor.

(24) Corticosteroids such as beclomethasone, methylprednisolone, betamethasone, prednisone, prednisolone, triamcinolone, dexamethasone, fluticasone, flunisolide, hydrocortisone, and corticosteroid analogs such as budesonide.

(25) Dietary supplements such as, for example omega-3 oils; folic acid, niacin, zinc, copper, Korean red ginseng root, ginkgo, pine bark, Tribulus terrestris, arginine, Avena sativa, horny goat weed, maca root, muira puama, saw palmetto, and Swedish flower pollen; vitamin C, Vitamin E, Vitamin K2; testosterone supplements, testosterone transdermal patch; zoraxel, naltrexone, bremelanotide and melanotan II.

(26) PGD2 receptor antagonists.

(27) Immunosuppressants such as cyclosporine (cyclosporine A, Sandimmune®, Neoral®), tacrolimus (FK-506, Prograf®), rapamycin (Sirolimus®, Rapamune®) and other FK-506 type immunosuppressants, mycophenolate, e.g., mycophenolate mofetil (CellCept®).

(28) Non-steroidal anti-asthmatics such as P2-agonists like terbutaline, metaproterenol, fenoterol, isoetharine, albuterol, salbutamol, salmeterol, bitolterol and pirbuterol; 2-agonist-corticosteroid combinations such as salmeterol-fluticasone (Advair®), formoterol-budesonide (Symbicort®), theophylline, cromolyn, cromolyn sodium, nedocromil, atropine, ipratropium, ipratropium bromide and leukotriene biosynthesis inhibitors (zileuton, BAY1005).

(29) Non-steroidal anti-inflammatory agents (NSAIDs) such as propionic acid derivatives like alminoprofen, benoxaprofen, bucloxic acid, carprofen, fenbufen, fenoprofen, fluprofen, flurbiprofen, ibuprofen, indoprofen, ketoprofen, miroprofen, naproxen, oxaprozin, pirprofen, pranoprofen, suprofen, tiaprofenic acid and tioxaprofen); acetic acid derivatives such as indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin and zomepirac; fenamic acid derivatives such as flufenamic acid, meclofenamic acid, mefenamic acid, niflumic acid and tolfenamic acid; biphenylcarboxylic acid derivatives such as diflunisal and flufenisal; oxicams such as isoxicam, piroxicam, sudoxicam and tenoxican; salicylates such as acetyl salicylic acid and sulfasalazine; and the pyrazolones such as apazone, bezpiperylon, feprazone, mofebutazone, oxyphenbutazone and phenylbutazone.

(30) Cyclooxygenase-2 (COX-2) inhibitors such as celecoxib (Celebrex®), rofecoxib (Vioxx®), valdecoxib, etoricoxib, parecoxib and lumiracoxib; opioid analgesics such as codeine, fentanyl, hydromorphone, levorphanol, meperidine, methadone, morphine, oxycodone, oxymorphone, propoxyphene, buprenorphine, butorphanol, dezocine, nalbuphine and pentazocine;

(31) Anti-diabetic agents such as insulin and insulin mimetics; sulfonylureas such as glyburide, glybenclamide, glipizide, gliclazide, gliquidone, glimepiride, meglinatide, tolbutamide, chlorpropamide, acetohexamide and olazamide; biguanides such as metformin (Glucophage®); a-glucosidase inhibitors such as acarbose, epalrestat, voglibose, miglitol; thiazolidinone compounds such as rosiglitazone (Avandia®), troglitazone (Rezulin®), ciglitazone, pioglitazone (Actos®) and englitazone; insulin sensitizers such as pioglitazone and rosiglitazone; insulin secretagogues such as repaglinide, nateglinide and mitiglinide; incretin mimetics such as exanatide and liraglutide; amylin analogues such as pramlintide; glucose lowering agents such as chromium picolinate, optionally combined with biotin; dipeptidyl peptidase IV inhibitors such as sitagliptin, vildagliptin, saxagliptin, alogliptin and linagliptin.

(32) HDL cholesterol-increasing agents such as anacetrapib and dalcetrapib.

(33) Antiobesity drugs such as methamphetamine hydrochloride, amfepramone hydrochloride (Tenuate®), phentermine (Ionamin®), benzfetamine hydrochloride (Didrex®), phendimetrazine tartrate (Bontril®, Prelu-2®, Plegine®), mazindol (Sanorex®), orlistat (Xenical®), sibutramine hydrochloride monohydrate (Meridia®, Reductil®), rimonabant (Acomplia®), amfepramone, chromium picolinate; combination such as phentermine/topiramate, bupropion/naltrexone, sibutramine/metformin, bupropion SR/zonisamide SR, salmeterol, xinafoate/fluticasone propionate; lorcaserin hydrochloride, phentermine/topiramate, cetilistat, exenatide, liraglutide, metformin hydrochloride, sibutramine/metformin, bupropion SR zonisamide SR, CORT-108297, canagliflozin, chromium picolinate, GSK-1521498, LY-377604, metreleptin, obinepitide, P-57AS3, PSN-821, salmeterol xinafoate/fluticasone propionate, sodium tungstate, somatropin (recombinant), tesamorelin, tesofensine, velneperit, zonisamide, beloranib hemioxalate, insulinotropin, resveratrol, sobetirome, tetrahydrocannabivarin and beta-lapachone. (34) Angiotensin receptor blockers such as losartan, valsartan, candesartan, cilexetil, eprosaran, irbesartan, telmisartan, olmesartran, medoxomil, azilsartan and medoxomil.

(35) Renin inhibitors such as aliskiren hemifumirate.

(36) Centrally acting alpha-2-adrenoceptor agonists such as methyldopa, clonidine and guanfacine.

(37) Adrenergic neuron blockers such as guanethidine and guanadrel.

(38) Imidazoline 1-1 receptor agonists such as rimenidine dihydrogen phosphate and moxonidine hydrochloride hydrate.

(39) Aldosterone antagonists such as spironolactone and eplerenone.

(40) Potassium channel activators such as pinacidil.

(41) Dopamine D1 agonists such as fenoldopam mesilate; other dopamine agonists such as ibopamine, dopexamine and docarpamine.

(42) 5-HT2 antagonists such as ketanserin.

(43) Vasopressin antagonists such as tolvaptan.

(44) Calcium channel sensitizers such as levosimendan or activators such as nicorandil

(45) PDE-3 inhibitors such as amrinone, milrinone, enoximone, vesnarinone, pimobendan, and olprinone.

(46) Adenylate cyclase activators such as colforsin dapropate hydrochloride.

(47) Positive inotropic agents such as digoxin and metildigoxin; metabolic cardiotonic agents such as ubidecarenone; brain natriuretic peptides such as nesiritide.

(48) Drugs used for the treatment of erectile dysfunction such as alprostadil, aviptadil, and phentolamine mesilate.

(49) Drugs used in the treatment of obesity, including but not limited to, methamphetamine hydrochloride (Desoxyn®), amfepramone hydrochloride (Tenuate®), phentermine (Lonamin®), benzfetamine hydrochloride (Didrex®), phendimetrazine hydrochloride (Bontril®, Prelu-2®, Plegine®), mazindol (Sanorex®) and orlistat (Xenical®).

(50) Drugs used for the treatment of Alzheimer's disease and dementias such as the following types: acetyl cholinesterase inhibitors including galantamine (Razadyne®), rivastigmine (Exelon®), donepezil (Aricept®) and tacrine (Cognex®); NMDA receptor antagonists such as memantine (Namenda®); and oxidoreductase inhibitors such as idebenone.

(51) Psychiatric medications such as the following types: ziprasidone (Geodon™), risperidone (Risperdal™), olanzapine (Zyprexa™), valproate; dopamine D4 receptor antagonists such as clozapine; dopamine D2 receptor antagonists such as nemonapride; mixed dopamine D1/D2 receptor antagonists such as zuclopenthixol; GABA A receptor modulators such as carbamazepine; sodium channel inhibitors such as lamotrigine; monoamine oxidase inhibitors such as moclobemide and indeloxazine; primavanserin, perospirone; and PDE4 inhibitors such as rolumilast.

(52) Drugs used for the treatment of movement disorders or symptoms such as the following types: catechol-O-methyl transferase inhibitors such as entacapone; monoamine oxidase B inhibitors such as selegiline; dopamine receptor modulators such as levodopa; dopamine D3 receptor agonists such as pramipexole; decarboxylase inhibitors such as carbidopa; other dopamine receptor agonists such as pergolide, ropinirole, cabergoline; ritigonide, istradefylline, talipexole; zonisamide and safinamide; and synaptic vesicular amine transporter inhibitors such as tetrabenazine.

(53) Drugs used for the treatment of mood or affective disorders or OCD such as the following types tricyclic antidepressants such as amitriptyline (Elavil®), desipramine (Norpramin®), imipramine (Tofranil®), amoxapine (Asendin®), nortriptyline and clomipramine; selective serotonin reuptake inhibitors (SSRIs) such as paroxetine (Paxil®), fluoxetine (Prozac®), sertraline (Zoloft®), and citralopram (Celexa®); doxepin (Sinequan®), trazodone (Desyrel®) and agomelatine; selective norepinephrine reuptake inhibitors (SNRIs) such as venlafaxine, reboxetine and atomoxetine; dopaminergic antidepressants such as bupropion and amineptine.

(54) Drugs for the enhancement of synaptic plasticity such as the following types: nicotinic receptor antagonists such as mecamylamine; and mixed 5-HT, dopamine and norepinephrine receptor agonists such as lurasidone.

(55) Drugs used for the treatment of ADHD such as amphetamine; 5-HT receptor modulators such as vortioxetine and alpha-2 adrenoceptor agonists such as clonidine.

(56) Neutral endopeptidase (NEP) inhibitors such as sacubitril, omapatrilat; and

(57) Methylene blue (MB).

Pharmaceutical Compositions and their Routes of Administration

The compounds herein disclosed, and their pharmaceutically acceptable salts, thereof may be formulated as pharmaceutical compositions or “formulations”.

A typical formulation is prepared by mixing a compound described herein, or a pharmaceutically acceptable salt thereof, and a carrier, diluent or excipient. Suitable carriers, diluents and excipients are well known to those skilled in the art and include materials such as carbohydrates, waxes, water soluble and/or swellable polymers, hydrophilic or hydrophobic materials, gelatin, oils, solvents, water, and the like. The particular carrier, diluent or excipient used will depend upon the means and purpose for which the compound described herein is being formulated. Solvents are generally selected based on solvents recognized by persons skilled in the art as safe (e.g., one described in the GRAS (Generally Recognized as Safe) database) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG400, PEG300), etc. and mixtures thereof. The formulations may also include other types of excipients such as one or more buffers, stabilizing agents, antiadherents, surfactants, wetting agents, lubricating agents, emulsifiers, binders, suspending agents, disintegrants, fillers, sorbents, coatings (e.g., enteric or slow release) preservatives, antioxidants, opaquing agents, glidants, processing aids, colorants, sweeteners, perfuming agents, flavoring agents and other known additives to provide an elegant presentation of the drug (i.e., a compound described herein or pharmaceutical composition thereof) or aid in the manufacturing of the pharmaceutical product (i.e., medicament).

The formulations may be prepared using conventional dissolution and mixing procedures. For example, the bulk drug substance (i.e., one or more of the compounds described herein, a pharmaceutically acceptable salt thereof, or a stabilized form of the compound, such as a complex with a cyclodextrin derivative or other known complexation agent) is dissolved in a suitable solvent in the presence of one or more of the excipients described above. A compound having the desired degree of purity is optionally mixed with pharmaceutically acceptable diluents, carriers, excipients or stabilizers, in the form of a lyophilized formulation, milled powder, or an aqueous solution. Formulation may be conducted by mixing at ambient temperature at the appropriate pH, and at the desired degree of purity, with physiologically acceptable carriers. The pH of the formulation depends mainly on the particular use and the concentration of compound, but may range from about 3 to about 8.

A compound described herein or a pharmaceutically acceptable salt thereof is typically formulated into pharmaceutical dosage forms to provide an easily controllable dosage of the drug and to enable patient compliance with the prescribed regimen. Pharmaceutical formulations of compounds described herein, or a pharmaceutically acceptable salt thereof, may be prepared for various routes and types of administration. Various dosage forms may exist for the same compound. The amount of active ingredient that may be combined with the carrier material to produce a single dosage form will vary depending upon the subject treated and the particular mode of administration. For example, a time-release formulation intended for oral administration to humans may contain approximately 1 to 1000 mg of active material compounded with an appropriate and convenient amount of carrier material which may vary from about 5 to about 95% of the total composition (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

The pharmaceutical compositions described herein will be formulated, dosed, and administered in a fashion, i.e., amounts, concentrations, schedules, course, vehicles, and route of administration, consistent with good medical practice. Factors for consideration in this context include the particular disorder being treated, the particular human or other mammal being treated, the clinical condition of the individual patient, the cause of the disorder, the site of delivery of the agent, the method of administration, the scheduling of administration, and other factors known to medical practitioners, such as the age, weight, and response of the individual patient.

The term “therapeutically effective amount” as used herein means that amount of active compound or pharmaceutical agent that elicits the biological or medicinal response in a tissue, system, animal or human that is being sought by a researcher, veterinarian, medical doctor or other clinician. The therapeutically effective amount of the compound to be administered will be governed by such considerations, and is the minimum amount necessary to ameliorate, cure or treat the disease or disorder or one or more of its symptoms.

The term “prophylactically effective amount” refers to an amount effective in preventing or substantially lessening the chances of acquiring a disorder or in reducing the severity of the disorder or one or more of its symptoms before it is acquired or before the symptoms develop further.

In some embodiments, a prophylactically effective amount of an NEP inhibitor is one that prevents or delays the occurrence, progression or reoccurrence of a gastrointestinal sphincter disorder. In further embodiments, a prophylactically effective amount of an NEP inhibitor is one that prevents or delays the occurrence or reoccurrence of symptoms in a subject suffering from a gastrointestinal sphincter disorder. In further embodiments, a prophylactically effective amount of an NEP inhibitor is one that prevents or delays the progression of an achalasia. In other embodiments, a prophylactically effective amount of an NEP inhibitor is one that prevents or delays the progression of dysphagia, esophageal aperistalsis, difficulty swallowing, regurgitation of undigested food, chest pain, cardiospasm, heartburn, shortness of breath, wheezing, cough, coughing when lying in a horizontal position, retention of food in the esophagus and aspiration of food into the lungs in a subject suffering a gastrointestinal sphincter disorder. In other embodiments, a prophylactically effective amount of an NEP inhibitor is one that prevents or delays the progression of dysphagia, esophageal aperistalsis, difficulty swallowing, regurgitation of undigested food, chest pain, cardiospasm, heartburn, shortness of breath, wheezing, cough, coughing when lying in a horizontal position, retention of food in the esophagus and aspiration of food into the lungs in a subject suffering an achalasia of a sphincter of the gastrointestinal tract.

Acceptable diluents, carriers, excipients, and stabilizers are those that are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). The active pharmaceutical ingredients may also be entrapped in microcapsules prepared, for example, by coacervation techniques or by interfacial polymerization, e.g., hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's: The Science and Practice of Pharmacy, 21^{st} Edition, University of the Sciences in Philadelphia, Eds., 2005 (hereafter “Remington's”).

“Controlled drug delivery systems” supply the drug to the body in a manner precisely controlled to suit the drug and the conditions being treated. The primary aim is to achieve a therapeutic drug concentration at the site of action for the desired duration of time. The term_“controlled release” is often used to refer to a variety of methods that modify release of drug from a dosage form. This term includes preparations labeled as “extended release”, “delayed release”, “modified release” or “sustained release”.

“Sustained-release preparations” are the most common applications of controlled release. Suitable examples of sustained-release preparations include semipermeable matrices of solid hydrophobic polymers containing the compound, which matrices are in the form of shaped articles, e.g. films, or microcapsules. Examples of sustained-release matrices include polyesters, hydrogels (for example, poly (2-hydroxy ethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers, and poly-D-(−)-3-hydroxybutyric acid.

“Gastroretentive formulations” are preparations designed to have increased retention in the stomach cavity. In some cases, they are used where a drug is preferentially or primarily absorbed via the stomach, is designed to treat the stomach directly, or where drug dissolution or absorption is aided drug absorption is aided by prolonged exposure to gastric acids. Examples of gastroretentive formulations include but are not limited to, high-density formulations, where the density of the formulation is higher than gastric fluid; floating formulations, which can float on top of gastric fluids due to increased buoyancy or lower density of the formulation; temporarily expandable formulations that are temporarily larger than the gastric opening; muco- and bio-adhesive formulations; swellable gel formulations; and in situ gel forming formulations. (See, e.g., Bhardwaj, L. et al. African J. of Basic & Appl. Sci. 4(6): 300-312 (2011)).

“Immediate-release preparations” may also be prepared. The objective of these formulations is to get the drug into the bloodstream and to the site of action as rapidly as possible. For instance, for rapid dissolution, most tablets are designed to undergo rapid disintegration to granules and subsequent disaggregation to fine particles. This provides a larger surface area exposed to the dissolution medium, resulting in a faster dissolution rate.

Implantable devices coated with a compound of this invention are another embodiment of the present invention. The compounds may also be coated on implantable medical devices, such as beads, or co-formulated with a polymer or other molecule, to provide a “drug depot”, thus permitting the drug to be released over a longer time period than administration of an aqueous solution of the drug. Suitable coatings and the general preparation of coated implantable devices are described in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition.

The formulations include those suitable for the administration routes detailed herein. The formulations may conveniently be presented in unit dosage form and may be prepared by any of the methods well known in the art of pharmacy. Techniques and formulations generally are found in Remington's. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

The terms “administer”, “administering” or “administration”, in reference to a compound, composition or formulation of the invention means introducing the compound into the system of the animal in need of treatment. When a compound of the invention is provided in combination with one or more other active agents, “administration” and its variants are each understood to include concurrent and/or sequential introduction of the compound and the other active agents.

The compositions described herein may be administered systemically or locally, e.g.: orally (e.g. using capsules, powders, solutions, suspensions, tablets, sublingual tablets and the like), by inhalation (e.g. with an aerosol, gas, inhaler, nebulizer or the like), to the ear (e.g. using ear drops), topically (e.g. using creams, gels, liniments, lotions, ointments, pastes, transdermal patches, etc.), ophthalmically (e.g. with eye drops, ophthalmic gels, ophthalmic ointments), rectally (e.g. using enemas or suppositories), nasally, buccally, vaginally (e.g. using douches, intrauterine devices, vaginal suppositories, vaginal rings or tablets, etc.), via an implanted reservoir or the like, or parenterally depending on the severity and type of the disease being treated. The term “parenteral” as used herein includes, but is not limited to, subcutaneous, intravenous, intramuscular, intraarticular, intra-synovial, intrasternal, intrathecal, intrahepatic, intralesional and intracranial injection or infusion techniques.

In particular embodiments, the compositions are administered orally, intraperitoneally or intravenously.

In other embodiments, the compositions are administered rectally.

The pharmaceutical compositions described herein may be orally administered in any orally acceptable dosage form including, but not limited to, capsules, tablets, aqueous suspensions or solutions. Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution-retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. Tablets may be uncoated or may be coated by known techniques including microencapsulation to mask an unpleasant taste or to delay disintegration and adsorption in the gastrointestinal tract and thereby provide a sustained action over a longer period. For example, a time delay material such as glyceryl monostearate or glyceryl distearate alone or with a wax may be employed. A water soluble taste masking material such as hydroxypropyl-methylcellulose or hydroxypropyl-cellulose may be employed.

Formulations of a compound described herein that are suitable for oral administration may be prepared as discrete units such as tablets, pills, troches, lozenges, aqueous or oil suspensions, dispersible powders or granules, emulsions, hard or soft capsules, e.g., gelatin capsules, syrups or elixirs. Formulations of a compound intended for oral use may be prepared according to any method known to the art for the manufacture of pharmaceutical compositions.

Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with a water-soluble carrier such as polyethylene glycol or an oil medium, for example, peanut oil, liquid paraffin, or olive oil.

The active compounds can also be in microencapsulated form with one or more excipients as noted above.

When aqueous suspensions are required for oral use, the active ingredient is combined with emulsifying and suspending agents. If desired, certain sweetening and/or flavoring agents may be added. Syrups and elixirs may be formulated with sweetening agents, for example glycerol, propylene glycol, sorbitol or sucrose. Such formulations may also contain a demulcent, a preservative, flavoring and coloring agents and antioxidant.

Sterile injectable forms of the compositions described herein (e.g., for parenteral administration) may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of injectable formulations.

Oily suspensions may be formulated by suspending a compound described herein in a vegetable oil, for example arachis oil, olive oil, sesame oil or coconut oil, or in mineral oil such as liquid paraffin. The oily suspensions may contain a thickening agent, for example, beeswax, hard paraffin or cetyl alcohol. Sweetening agents such as those set forth above, and flavoring agents may be added to provide a palatable oral preparation. These compositions may be preserved by the addition of an anti-oxidant such as butylated hydroxyanisol or alpha-tocopherol.

Aqueous suspensions of compounds described herein contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable drug-depot forms are made by forming microencapsulated matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Drug-depot injectable formulations are also prepared by entrapping the compound in liposomes or microemulsions that are compatible with body tissues.

The injectable solutions or microemulsions may be introduced into a patient's bloodstream by local bolus injection. Alternatively, it may be advantageous to administer the solution or microemulsion in such a way as to maintain a constant circulating concentration of the instant compound. In order to maintain such a constant concentration, a continuous intravenous delivery device may be utilized. An example of such a device is the Deltec CADD-PLUS™ model 5400 intravenous pump.

Compositions for rectal or vaginal administration are preferably suppositories which can be prepared by mixing the compounds described herein with suitable non-irritating excipients or carriers such as cocoa butter, beeswax, polyethylene glycol or a suppository wax which are solid at ambient temperature but liquid at body temperature and therefore melt in the rectum or vaginal cavity and release the active compound. Other formulations suitable for vaginal administration may be presented as pessaries, tampons, creams, gels, pastes, foams or sprays.

The pharmaceutical compositions described herein may also be administered topically, especially when the target of treatment includes areas or organs readily accessible by topical application, including diseases of the eye, the ear, the skin, or the lower intestinal tract. Suitable topical formulations are readily prepared for each of these areas or organs.

Dosage forms for topical or transdermal administration of a compound described herein include ointments, pastes, creams, lotions, gels, powders, solutions, sprays, inhalants or patches.

The active component is admixed under sterile conditions with a pharmaceutically acceptable carrier and any needed preservatives or buffers as may be required. Ophthalmic formulation, eardrops, and eye drops are also contemplated as being within the scope of this invention.

Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel. Topical application for the lower intestinal tract can be effected in a rectal suppository formulation (see above) or in a suitable enema formulation. Topically-transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl ester wax, cetearyl alcohol, 2 octyldodecanol, benzyl alcohol and water.

For ophthalmic use, the pharmaceutical compositions may be formulated as micronized suspensions in isotonic, pH-adjusted sterile saline, or, preferably, as solutions in isotonic, pH-adjusted sterile saline, either with or without a preservative such as benzylalkonium chloride. Alternatively, for ophthalmic uses, the pharmaceutical compositions may be formulated in an ointment such as petrolatum. For treatment of the eye or other external tissues, e.g., mouth and skin, the formulations may be applied as a topical ointment or cream containing the active ingredient(s) in an amount of, for example, between 0.075% and 20% w/w. When formulated in an ointment, the active ingredients may be employed with either an oil-based, paraffinic or a water-miscible ointment base.

Alternatively, the active ingredients may be formulated in a cream with an oil-in-water cream base. If desired, the aqueous phase of the cream base may include a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulfoxide and related analogs.

The oily phase of emulsions prepared using compounds described herein may be constituted from known ingredients in a known manner. While the phase may comprise merely an emulsifier (otherwise known as an emulgent), it desirably comprises a mixture of at least one emulsifier with a fat or an oil or with both a fat and an oil. A hydrophilic emulsifier may be included together with a lipophilic emulsifier which acts as a stabilizer. In some embodiments, the emulsifier includes both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulgents and emulsion stabilizers suitable for use in the formulation of compounds described herein include Tween™-60, Span™-80, cetostearyl alcohol, benzyl alcohol, myristyl alcohol, glyceryl mono-stearate and sodium lauryl sulfate.

The pharmaceutical compositions may also be administered by nasal aerosol or by inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents. Formulations suitable for intrapulmonary or nasal administration may have a mean particle size in the range of, for example, 0.1 to 500 microns (including particles with a mean particle size in the range between 0.1 and 500 microns in increments such as 0.5, 1, 30, 35 microns, etc.), which may be administered by rapid inhalation through the nasal passage or by inhalation through the mouth so as to reach the alveolar sacs.

The pharmaceutical composition (or formulation) for use may be packaged in a variety of ways depending upon the method used for administering the drug. Generally, an article for distribution includes a container having deposited therein the pharmaceutical formulation in an appropriate form. Suitable containers are well-known to those skilled in the art and include materials such as bottles (plastic and glass), sachets, ampoules, plastic bags, metal cylinders, and the like. The container may also include a tamper-proof assemblage to prevent indiscreet access to the contents of the package. In addition, the container has deposited thereon a label that describes the contents of the container. The label may also include appropriate warnings.

The formulations may be packaged in unit-dose or multi-dose containers, for example sealed ampoules and vials, and may be stored in a freeze-dried (lyophilized) condition requiring only the addition of the sterile liquid carrier, for example water, for injection immediately prior to use.

Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient. In another aspect, a compound described herein or a pharmaceutically acceptable salt, co-crystal, solvate or pro-drug thereof may be formulated in a veterinary composition comprising a veterinary carrier. Veterinary carriers are materials useful for the purpose of administering the composition and may be solid, liquid or gaseous materials which are otherwise inert or acceptable in the veterinary art and are compatible with the active ingredient. These veterinary compositions may be administered parenterally, orally or by any other desired route.

### Kits

The pharmaceutical formulations described herein may be contained in a kit. The kit may include single or multiple doses of two or more agents, each packaged or formulated individually, or single or multiple doses of two or more agents packaged or formulated in combination. Thus, one or more agents can be present in first container, and the kit can optionally include one or more agents in a second container. The container or containers are placed within a package, and the package can optionally include administration or dosage instructions. A kit can include additional components such as syringes or other means for administering the agents as well as diluents or other means for formulation. Thus, the kits can comprise: a) a pharmaceutical composition comprising a compound described herein and a pharmaceutically acceptable carrier, vehicle or diluent; and b) another therapeutic agent and a pharmaceutically acceptable carrier, vehicle or diluent in one or more containers or separate packaging. The kits may optionally comprise instructions describing a method of using the pharmaceutical compositions in one or more of the methods described herein (e.g. preventing or treating one or more of the diseases and disorders described herein). The pharmaceutical composition comprising the compound described herein and the second pharmaceutical composition contained in the kit may be optionally combined in the same pharmaceutical composition.

A kit includes a container or packaging for containing the pharmaceutical compositions and may also include divided containers such as a divided bottle or a divided foil packet. The container can be, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. It is feasible that more than one container can be used together in a single package to market a single dosage form. For example, tablets may be contained in a bottle which is in turn contained within a box.

An example of a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process, recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening. It may be desirable to provide written memory aid containing information and/or instructions for the physician, pharmacist or subject regarding when the medication is to be taken. A “daily dose” can be a single tablet or capsule or several tablets or capsules to be taken on a given day. When the kit contains separate compositions, a daily dose of one or more compositions of the kit can consist of one tablet or capsule while a daily dose of another one or other compositions of the kit can consist of several tablets or capsules. A kit can take the form of a dispenser designed to dispense the daily doses one at a time in the order of their intended use. The dispenser can be equipped with a memory-aid, so as to further facilitate compliance with the regimen. An example of such a memory-aid is a mechanical counter which indicates the number of daily doses that have been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

### EXAMPLES

### Example 1. Non-Clinical Studies

In vivo mouse models: A transgenic rat model (Pvrl3-Cre) of achalasia has recently been developed and described (“Megaesophagus in a line of transgenic rats: a model of achalasia”; Pang J; Borjeson T M; Muthupalani S; Ducore R M; Carr C A; Feng Y; Sullivan M P; Cristofaro V; Luo J; Lindstrom J M; Fox J G; Veterinary pathology, 51(6): 1187-200, 2014). These rats present with an abnormal enlargement of the esophagus at 3 to 4 months of age and a reduced number of myenteric neurons leading to features similar to human disease. The utility of an NEP inhibitor to treat achalasia could be assessed in a study utilizing these rats. 4-week-old Pvrl3-Cre rats would be divided into groups of 10-12 rats per treatment group and would receive NEP inhibitor over the course of 7 weeks. Rats would be dosed with an NEP inhibitor by oral gavage (ranging from 1 to 10 mg/kg/day, qd or bid) or by administration of an equivalent dose in food. One group would serve as a vehicle control. Relevant endpoints would be body weight, assessment of the esophagus and lower esophageal sphincter by contrast radiography and fluoroscopy, and histological assessment of the esophagus including the number of myenteric neurons. An NEP inhibitor would be expected to preserve body weight, normalize enlargement of the esophagus, and normalize esophageal function.

Ex vivo models: The effect of NEP inhibitor on muscle contractility would be measured in ex vivo studies on lower esophageal sphincter tissue isolated from rats. The lower esophageal sphincter would be isolated from the esophagus of a rat and strips of circular smooth muscle tissue would be prepared. The tissue strip would be suspended under tension in an organ bath and the mechanical force of the tissue would be determined using an isometric force transducer. Simultaneous measurement of multiple isolated tissues from the same sphincter from the same donor would be conducted over the course of the study. The tissue would be subjected to a steady and consistent tension and then treated with carbachol to induce a contraction. The ability of an NEP inhibitor to induce relaxation of carbachol-induced contraction would be determined as follows:
Vehicle
PDE 5 inhibitor (cumulative concentrations)
NEP inhibitor (cumulative concentrations ranging from 1 nM to 100 uM)
Sub-threshold concentration of PDE 5 inhibitor+NEP inhibitor (1 nM to 10 uM)

Both PDE 5 inhibitor and NEP inhibitor would be expected to relax esophageal smooth muscle and act together in an additive or synergistic fashion.

### Example 2. Clinical Studies

### Clinical Studies—A

The effect of NEP inhibitors can be determined clinically in human patients with idiopathic achalasia by manometry—a measure of the esophageal pressure gradient in response to swallowing. PDE5 inhibitors, such as sildenafil, which similarly result in increased levels of cGMP, have been used off label in achalasia patients and have shown some limited utility (“Effects of sildenafil on esophageal motility of patients with idiopathic achalasia”; Bortolotti M; Mari C; Lopilato C; Porrazzo G; Miglioli M; Gastroenterology, 118(2): 253-7, 2000). Achalasia patients would be fasted overnight and then prepped in the morning with a manometric pressure probe. NEP inhibitors would be administered p.o. Patients would then be asked to perform dry swallows at approximately 30-60-second intervals for the entire recording period while manometric pressure would be measured. An NEP inhibitor would be expected to reduce esophageal pressure, induce relaxation of the lower esophageal sphincter, and restore esophageal peristalsis.

### Clinical Studies—B

A multicenter, randomized, double-blind, placebo-controlled, parallel-group, single-dose study will randomize approximately 20 patients to receive an NEP inhibitor of the invention (15 patients to NEP inhibitor and 5 to matching placebo). The study will randomize patients diagnosed with type II achalasia with an integrated relaxation pressure (IRP)>15 mm Hg by baseline high resolution impedance manometry (HRIM).

Test product (an NEP inhibitor as described above) will be administered orally as 1 mg tablets; the dose will be a total of 5 mg (5 tablets). Placebo will match the NEP inhibitor oral tablets. Patients will begin a liquid diet on Day−1 and then will fast overnight. To confirm eligibility, patients will undergo a baseline protocol-specific HRIM procedure that includes 2 swallowing sequences recorded 1 hour (±15 minutes) apart.

After the second recording, the HRIM catheter will be removed, and patients will complete a baseline symptom assessment. Patients who meet all eligibility criteria in addition to having confirmed type II achalasia and IRP>15 mm Hg will be randomized to receive a single 5-mg dose of the NEP inhibitor or matching placebo, together with 8 oz. of water. Following study drug administration, the HRIM catheter will be reinserted for the post-dose HRIM procedure. The HRIM catheter will be removed after the final recording, and patients will complete a post-dose symptom assessment.

### Other Embodiments

All publications and patents referred to in this disclosure are incorporated herein by reference to the same extent as if each individual publication or patent application were specifically and individually indicated to be incorporated by reference. Should the meaning of the terms in any of the patents or publications incorporated by reference conflict with the meaning of the terms used in this disclosure, the meaning of the terms in this disclosure are intended to be controlling. Furthermore, the foregoing discussion discloses and describes merely exemplary embodiments of the present invention. One skilled in the art will readily recognize from such discussion and from the accompanying drawings and claims, that various changes, modifications and variations can be made therein without departing from the spirit and scope of the invention as defined in the following claims.

What is claimed is:

1. A method of treating a gastrointestinal sphincter disorder in a patient in need thereof, comprising administering to said patient a therapeutically effective amount of neural endopeptidase (NEP) inhibitor or a pharmaceutically acceptable salt thereof.

2. The method of claim 1, wherein the gastrointestinal sphincter disorder is an achalasia of a sphincter of the gastrointestinal tract.

3. The method of claim 1, wherein the gastrointestinal sphincter disorder is a spastic sphincter disorder of the gastrointestinal tract or sphincter spasms.

4. The method of claim 1, wherein the gastrointestinal sphincter disorder is a hypertensive sphincter disorder of the gastrointestinal tract.

5. The method of any one of claims 1 to 4, wherein the gastrointestinal sphincter is selected from lower esophageal sphincter (LES), pyloric sphincter (pylorus), ileocecal sphincter or valve (ICV), the sphincter of Oddi (SO, also named Glisson's sphincter) and internal anal sphincter (IAS).

6. The method of claim 2, wherein the achalasia of a sphincter of the gastrointestinal tract is esophageal achalasia.

7. The method of claim 2, wherein the achalasia of a sphincter of the gastrointestinal tract is a primary achalasia.

8. The method of claim 2, wherein the achalasia of a sphincter of the gastrointestinal tract is a secondary achalasia.

9. The method of claim 6, wherein the achalasia is primary esophageal achalasia.

10. The method of claim 6, wherein the achalasia is secondary esophageal achalasia.

11. The method of claim 8 or claim 10, wherein the achalasia is a secondary achalasia associated with Chagas disease.

12. The method of claim 10, wherein the achalasia is secondary esophageal achalasia associated with esophageal cancer.

13. The method of claim 1, wherein the gastrointestinal sphincter disorder is selected from, lower esophageal sphincter (LES) achalasia, esophageal achalasia, spastic LES, hypertensive LES (HTNLES), pyloric sphincter (pylorus) achalasia, pyloric spasm (pylorospasm), hypertensive pylori, ileocecal sphincter or valve (ICV) achalasia, hypertensive ICV, spastic ICV or ICV spasm, sphincter of Oddi dysfunction (SOD), spastic sphincter of Oddi or SOD spasm, hypertensive sphincter of Oddi, hypertensive IAS, spastic IAS or IAS spasm.

14. The method of any one of claims 1 to 13, wherein the gastrointestinal sphincter disorder is a secondary gastrointestinal sphincter disorder associated with diabetes, systemic sclerosis, Chagas disease, a neurodegenerative or neurological disease, brain, head or neck injury or trauma or a paraneoplastic syndrome.

15. The method of any one of claims 1 to 14, wherein said NEP inhibitor or pharmaceutically acceptable salt thereof is administered as a monotherapy.

16. The method of any one of claims 1 to 14, wherein said NEP inhibitor or pharmaceutically acceptable salt thereof is administered in combination with a therapeutically or prophylactically effective amount of one or more additional therapeutic agents.

17. The method of claim 16, wherein the additional therapeutic agent is a PDE inhibitor.

18. The method of claim 17, wherein the additional therapeutic agent is a PDE5 inhibitor.

19. The method of claim 18, wherein the PDE5 inhibitor is selected from the group consisting of sildenafil, avanafil, lodenafil, mirodenafil, sildenafil citrate, tadalafil, vardenafil, udenafil, alprostadil, dipyridamole, and PF-00489791.

20. The method of any one of claims 16 to 19, wherein the NEP inhibitor is administered prior to, at the same time as, or after the initiation of treatment with the additional therapeutic agent.

21. The method of any one of claims 1 to 20, wherein the NEP inhibitor is selected from sacubitril, TD-1439, TD-0714, TD-0212, daglutril, ilepatril, SLV-338, UK-447841, VPN-489, LBQ657 and LHW-090.

22. The method of any one of claims 1 to 21 wherein the patient in need thereof is an adult.

23. The method of any one of claims 1 to 21, wherein the patient in need thereof is a child.

24. A pharmaceutical composition comprising an NEP inhibitor, or a pharmaceutically acceptable salt thereof, for use in the treatment of a gastrointestinal sphincter disorder in a patient in need thereof.

25. A pharmaceutical composition comprising an NEP inhibitor, or a pharmaceutically acceptable salt thereof, and one or more additional therapeutic agents, for use in the treatment of a gastrointestinal sphincter disorder in a patient in need thereof.
